(19) United States
(12) Patent Application Publication
(10) Pub. No.: US 2013/0123821 A1
(43) Pub. Date: May 16, 2013

(54) Single use, multipurpose neuro-dissector

(71) Applicants: John Cobb, Lafayette, LA (US); William Atkinson, Lafayette, LA (US)

(72) Inventors: John Cobb, Lafayette, LA (US); William Atkinson, Lafayette, LA (US)

(21) Appl. No.: 13/294,333

(22) Filed: Nov. 11, 2011

(51) Int. Cl. A61B 17/32

(52) U.S. Cl. 606/170

(57) ABSTRACT

A single-use, multi-purpose neuro-dissector (“dissector”), suitable for a variety of surgical procedures, is disclosed. A handle of a material which degrades in heat sufficient to sterilize same holds an extended, slender rod, preferably of stainless steel or similar suitable material. A section of the rod near its distal end (away from the handle) is turned at an angle to the remainder of the rod, and a cutting surface and cutting edge are formed in the turned-down section. The distal end of the rod forms a non-cutting tip, for example a blunt ball tip, to avoid unintentional cutting of surrounding tissues. The cutting surface and edge are coated with a material which prevents re-sharpening, such as titanium nitride. The combination of a handle material which degrades in the heat of sterilization, if attempted, and a cutting surface which is not re-sharpenable, render the dissector a single-use device.

### BACKGROUND

1. Field of the Invention

This invention relates, generally, to apparatus and methods used in surgical procedures, generally (although not necessarily restricted to) surgery of different types on human patients. More specifically, the invention relates to a type of single-use scalpel or surgical instrument, namely a neuro-dissector, having design and material attributes which prevent multiple use.

2. Background

Surgical instruments of many sorts are known in the art. In particular, various types of surgical instruments are used in neurological and similar procedures. Such surgical procedures require dissection of certain tissue, while at the same time requiring protection against dissection of surrounding tissue. It is important that the surgeon be able to readily visualize the tissues being dissected, in order to properly guide the surgical instrument. Types of surgical procedures which require neurological dissection include, but are not limited to:
carpal and tarsal tunnel release
incision of ligamentum flavum during cervical and lumbar laminectomies
dissection of the arachnoid in cranial procedures
release of posterior longitudinal ligament in anterior cervical procedures to facilitate exposure to the epidural space and neuroforamen
release of the posterior longitudinal ligament in anterior lumbar procedures, for example soft tissue balancing in total disc replacements
neurolysis in revision spinal procedures
neurolysis in peripheral nerve surgery

Yet another issue arises in connection with these types of surgical procedures, and the instruments used to carry them out. Sterilization and sharpening of multi-use instruments used in neurological procedures becomes problematic. With regard to sterilization, as is well known, multi-use surgical instruments must be sterilized to a high sterility assurance level (known as SAL) between uses. While various methods of sterilization exist, probably the most common is steam sterilization at a combination of high temperature (typically 250 F to 273 F) and pressure, applied in an autoclave, with the surgical instrument present for a sufficient length of time (typically in the range of 3 to 15 minutes, depending upon temperature). Despite best practices, there is the ever-present risk of inadequate sterilization procedures being carried out on multi-use devices, as opposed to single-use devices that can be manufactured, packaged, and shipped in a sterile environment and packaging, only opened by the surgical staff at the time of use. With regard to sharpening, it can be readily appreciated that surgical instruments must be of the utmost sharpness. The very small cutting surfaces on many of the dissectors used in neurological procedures are frequently difficult to effectively resharpen, hence reuse presents issues associated with insufficiently sharpened instruments.

### SUMMARY

Known neuro-dissector instruments present various limitations due to their structure and intended use as multi-use instruments. The present invention comprises structural attributes which permit efficient and safe dissection of neurological tissues, and is a single use instrument, which must be discarded after one use.

Accordingly, the neuro-dissector has an elongated handle which affords a comfortable and secure grip for the surgeon. The handle is preferably made of a material which will degrade when subjected to heat sterilization techniques. While various materials may be possible, one suitable material is a low density polyethylene plastic which degrades with heat. Thus, subjecting the handle to sterilization by heat will result in its destruction, and renders the dissector unusable. Preferably, the overall contour of the handle includes a rounded, larger diameter section nearest one end, with a smaller, uniform diameter section proximal the opposite end. A non-slip surface may be provided on the handle, for example a number of ridges, ribs or fins forming the outer diameter of the handle.

The neuro-dissector handle holds an extended rod or similar shaped member, with a section proximate the distal end (the end farthest from the handle) turned down to form the cutting surface and cutting edge. Preferably, the rod is attached to the larger diameter end of the handle. The cutting surface and cutting edge is generally aligned at a large angle, for example a seventy degree angle, to the longitudinal axis of the rod. This alignment means that the cutting action for the neuro-dissector is generally a pulling action on the instrument. The rod terminates in a blunt ball tip, which is non-cutting and provides protection to the target neural tissue. The alignment of the handle, rod, and cutting edge are such that the surgeon can maintain full visualization during surgery.

The cutting edge of the dissector is also designed to ensure single use. The rod, including the cutting edge, is preferably of stainless steel. The actual cutting edge is coated with a substance which is extremely hard and destructive to sharpening devices, thereby ensuring that the cutting edge will not be re-sharpened and enforcing the single-use aspect of the device. One suitable cutting surface and cutting edge coating material is titanium nitride (TiN).

### BRIEF DESCRIPTION OF THE DRAWINGS

FIG. 1 is a perspective view of the surgical instrument embodying the principles of the invention.

FIG. 2 is a view from the handle portion of the instrument, looking toward the cutting edge of the instrument.

FIG. 3 is a detail view of the distal end section of the elongated rod, and the cutting surface and cutting edge.

FIG. 4 is a side view of the surgical instrument embodying the principles of the invention.

FIG. 5 is another side view of the surgical instrument, rotated 90 degrees about its longitudinal axis from the view of FIG. 4.

### DETAILED DESCRIPTION OF THE PRESENTLY PREFERRED EMBODIMENT(S)

While the apparatus may take various forms, all falling within the scope of the present invention, with reference to the figures some of the presently preferred embodiments will now be described.

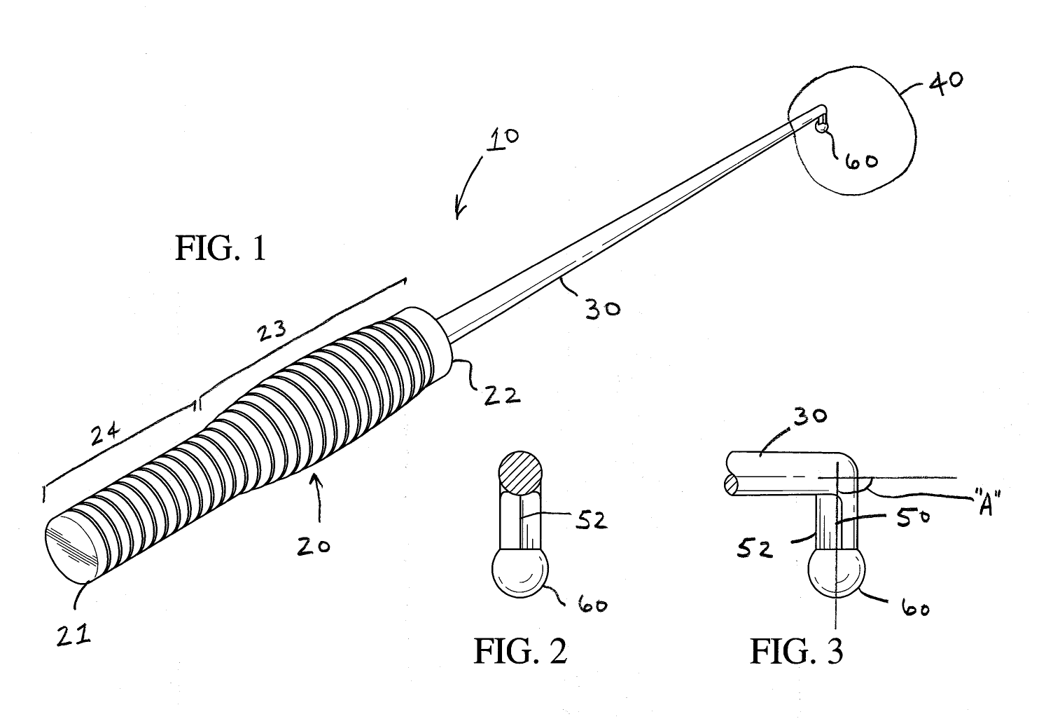

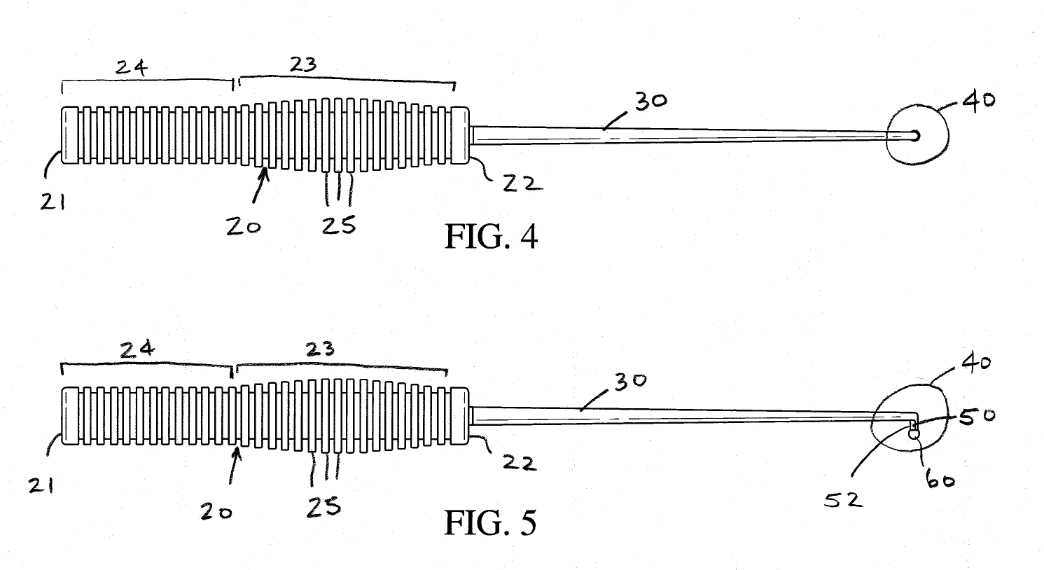

The surgical instrument of the present invention, namely a single use, multi-purpose neuro-dissector 10, referred to at times as dissector 10, comprises an elongated handle 20 with first and second ends 21 and 22 and an extended rod 30 fixed to and extending outwardly from handle 20, with a distal end section 40, as readily seen in FIGS. 1, 4 and 5. Preferably, the overall contour of handle 20 includes a rounded, larger diameter section 23 proximal second end 22, with a smaller, uniform diameter section 24 proximal first end 21; these attributes are readily seen when viewing dissector 10 from the side, as in FIGS. 4 and 5. A non-slip surface may be provided on the handle, for example a number of ridges, ribs or fins 25 forming the outer diameter of handle 20. Dimensions of handle 20 are of a size (and shape) suitable for grasping by a surgeon, and may be varied to suit the user, with a length of approximately 4 inches suitable for many users. Diameter of handle 20 may be approximately 0.75″ at its maximum point in larger diameter section 23, and a uniform 0.4″ over smaller diameter section 24, although other dimensions are suitable.

With regard to rod 30, distal end section 40 is bent from the longitudinal axis of rod 30 at a suitable angle to form a cutting surface 50, as seen in FIGS. 1, 4 and 5, and in detail in FIGS. 2 and 3. The distal end (the end farthest from handle 20) of rod 30 terminates in a blunt, non-cutting tip, such as blunt ball tip 60. As can be appreciated, cutting surface 50 is disposed at an angle with the longitudinal axis of rod 30, with edge 52 oriented back toward handle 20, such that cutting of tissue is generally effected by a pulling motion of dissector 10 toward the surgeon. FIG. 2 is a view from the handle portion looking toward the cutting edge 52. The angle of cutting surface 50 (and cutting edge 52) with the longitudinal axis of rod 30 (as measured and shown generally as angle “A” in FIG. 3) may be on the order of 70 degrees, although larger or smaller angles are possible. Blunt tip 60, which is generally in a ball or similar shape, permits the dissector to be inserted between areas of tissue, without cutting same.

Rod 30 may be joined to handle 20 by threading, adhesively joining, or other methods well known in the art. If desired, rod 30 may be fixed to handle 20 such that the longitudinal axis of rod 30 forms an angle with the longitudinal axis of handle 20, to permit complete visualization of the cutting surface and the tissues being cut, by the surgeon. While various angles may be suitable, from about 0 to 20 degrees, the angle is preferably about 10-12 degrees, depending upon the line of sight of the individual user. Rod 30 is preferably made of stainless steel or other suitable material, with a length beyond handle 20 of approximately 4 inches and a base diameter of 1/8 inch, tapering toward the distal end section. It is understood that dimensions and materials may be changed to suit the user. One suitable material for rod 30 and cutting surface 50 is stainless steel, grade 17-4 (as specified in ASTM A564/A564M-01).

The materials for handle 20 and cutting surface 50 are important for the single-use aspect of dissector 10. Handle 20, generally, is of a material which will degrade with heat and pressure to such an extent that dissector 10 would be unusable, if heat/pressure sterilization were attempted. While various materials may be suitable, one presently preferred material comprises a low density polyethylene plastic, which will substantially degrade with heat and pressure. It is understood that other materials may be possible.

In addition to the attributes of handle 20, cutting surface 50 and edge 52 are coated with a substance which is very hard and destructive to sharpening devices, thereby ensuring that the cutting surface 50 and edge 52 will not be re-sharpened, and enforcing the single-use aspect of the device. One suitable cutting surface coating material is titanium nitride (sometimes referred to in the art as TiN). It is known in the art that TiN is an extremely hard ceramic material, often used to harden and protect cutting surfaces. It is not readily re-sharpenable, in fact is sufficiently hard that it would be destructive to sharpening devices. It is understood that other materials may be suitable for coating of cutting surface 50 and edge 52, as long as they possess properties similar to those of TiN.

### CONCLUSION

While the preceding description contains many specificities, it is to be understood that same are presented only to describe some of the presently preferred embodiments of the invention, and not by way of limitation. Changes can be made to various aspects of the invention, without departing from the scope thereof. For example:
dimensions of dissector 10 may be varied to suit the user
the angle of rod 30 with respect to handle 20 may be varied
the shape of cutting surface 50 and edge 52, and their angle with respect to rod 30, may be varied to suit the user
materials for handle 20 may be any material which degrades to a significant degree with heat and pressure, including various thermoplastics
coating and other treatment of cutting surface 50 and edge 52 may be of different materials, as long as same are non-sharpenable due to hardness or other attributes

Therefore, the scope of the invention is to be determined not by the illustrative examples set forth above, but by the appended claims and their legal equivalents.

I claim:

1. A surgical instrument, comprising:
an elongated handle having a size and shape suitable for grasping by a surgeon;
an elongated rod attached to said handle and extending therefrom, said elongated rod having a distal end section distal from said handle, said distal end section turned at an angle to a longitudinal axis of said rod, a cutting surface formed in said distal end section and having a cutting edge generally facing said handle, said rod terminating in a non-cutting tip,
wherein said handle is formed from a material which degrades when subjected to a heat and pressure environment sufficient to sterilize same, said degradation sufficient to render said surgical device unusable, and wherein said cutting surface and edge comprise a material having characteristics which prevent re-sharpening thereof,
whereby after a single surgical procedure, said surgical instrument is disposed of.

2. The surgical instrument of claim 1, wherein said handle is formed from a low-density polyethylene; and
said cutting surface and edge are coated with titanium nitride.

3. The surgical instrument of claim 2, wherein said handle comprises a cross section shape when viewed from a side comprising a first, larger diameter section proximal said rod, tapering down to a uniform dimension section on an end distal from said rod.

4. The surgical instrument of claim 3, wherein said handle comprises a plurality of outwardly extending fins forming said cross section shape.

5. A single use, multipurpose neuro-dissector, comprising:
an extended handle;
a rod attached to and extending from said handle, said rod having a distal section positioned away from said handle, said distal section turned at an angle to a longitudinal axis of said rod, said distal section comprising a cutting surface and cutting edge oriented toward said handle, said rod terminating in a blunt tip;
wherein said handle, when exposed to a temperature sufficiently high to sterilize said handle, degrades to such an extent that said handle is unusable; and
wherein said cutting surface is coated with a material of sufficient hardness that said cutting surface cannot be re-sharpened.

6. The dissector of claim 5, wherein said handle is formed from a low-density polyethylene.

7. The dissector of claim 5, wherein said cutting surface is coated with titanium nitride.

8. The dissector of claim 5, wherein said handle is formed from a low-density polyethylene, and wherein said cutting surface is coated with titanium nitride.

9. The dissector of claim 8, wherein said handle comprises a larger diameter section proximal said rod, and a smaller diameter remaining section.
